(19) United States
(12) Patent Application Publication
(10) Pub. No.: US 2005/0214236 A1
(43) Pub. Date: Sep. 29, 2005

(54) Surface treated pearlescent pigments for hair care products

(76) Inventors: Qinyun Peng, Yorktown Heights, NY (US); Philip Linz, Croton-Hudson, NY (US); Mark Tellefsen, Savannah, GA (US); Francois Marchio, Scarsdale, NY (US); Ratan C. Chaudhuri, Lincoln Park, NJ (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/018,932

(22) Filed: Dec. 22, 2004

Related U.S. Application Data
(60) Provisional application No. 60/531,625, filed on Dec. 23, 2003.

(51) Int. Cl. C04B 2/00; A61K 7/06

(52) U.S. Cl. 424/70.1; 106/415; 106/417; 106/436; 106/499

(57) ABSTRACT

A surface treated pearlescent pigment obtained by a process comprising hydrolyzing an amine of the formulae
(R)₂N—(CH₂)ₙ)—SIR′ R=alkyl or aryl
or
A⁻(R)₃—N—(CH₂)ₙ—SiR′ R′=alkoxy, C₁-C₃ n=3-5
A⁻=an anion to obtain silanol groups and contacting a pearlescent pigment with resultant hydrolyzate.

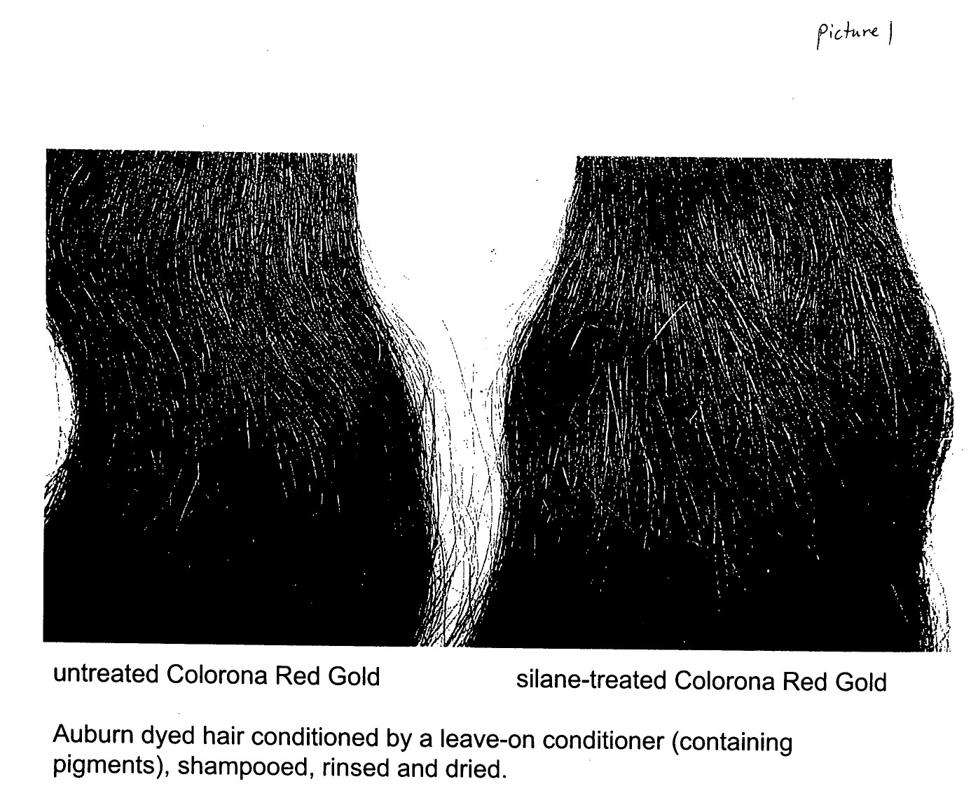

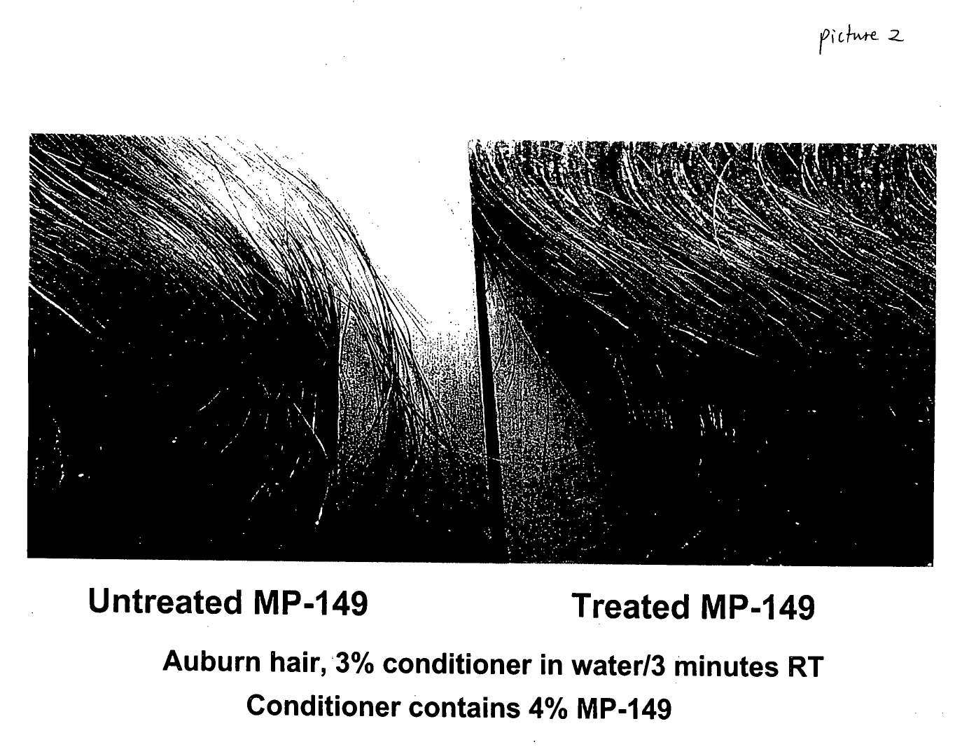

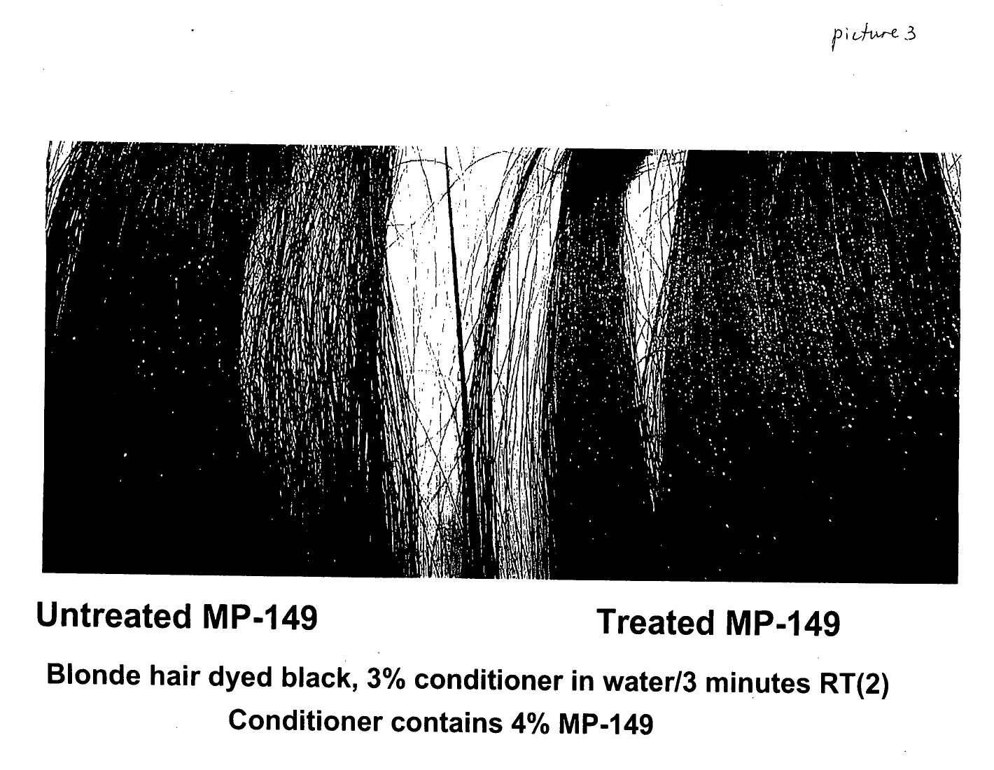

Applicants claim the benefit of Provisional Application Ser. No. 60/531,625 filed Dec. 23, 2003, and is incorporated by reference herein.

### BACKGROUND

It has been known that a surface treated pearlescent pigment can have certain advantages for specific applications.

U.S. Pat. Nos. 5,472,491, 5,759,255 and 6,176,918 disclose the surface treatment of pearlescent pigments with coupling agents, such as silane, zirconium aluminates. The treated materials are used for exterior solvent-borne or water-borne applications. U.S. Pat. No. 6,488,757 teaches the surface treatment of pearlescent pigments with organic hydrophobic coupling agents to provide hydrophobicity to the pigments.

WO 03/043567 indicates that pigments treated with polysiloxanes have improved water repellency, no outgassing, improved stability and smooth feel and adhesion to skin when they are used in cosmetic formulations. The attributes of the treated pigments result from materials with a polysiloxane backbone. Their applications are powder formulations, oil-in water emulsions, water-in-oil emulsions, anhydrous make-up and lipstick.

### THE INVENTION

The present invention describes the surface treatment of pearlescent pigments with silane for cosmetics and personal care products, particularly for hair care products with resultant improved substativity.

A specific type of silane product was chosen to treat the pearlescent pigments in order to improve the affinity of pearlescent pigments to hair and skin in cosmetic products. The general structure of the chosen silanes includes at least one amino group, specifically, a tertiary or quaternary amine, connected to straight or branched chain of C₃₋₅, silicon and silicon functional groups (see below).
(R)₂N—(CH₂)ₙ—Si(R′)₃ R=alkyl or aryl
A⁻(R)₃N—(CH₂)ₙ—Si(R′)₃ R′=alkoxy, C₁-C₃
n=3-5
A⁻=an anion

In the above formulae, R preferably contains 1-18 carbon atoms, and preferably represents alkyl, e.g. methyl and decyl; R′ is preferably methoxy; n is preferably 3; and A⁻ is preferably Cl⁻. Particularly preferred silanes include but are not limited to:
dimethylaminopropyltrimethoxysilane,
didecylmethyl(trimethoxysilylpropyl)ammonium,
chloride and tri-methyl(trimethoxysylilpropyl)ammonium chloride.

The pigments used in this invention are pearlescent pigments, such as platelet-shaped substrates coated with metal oxides, bismuth oxychloride particles, or other platelet-shaped pearlescent pigments, such as titanium dioxide platelets, iron oxide platelets. For pigments with platelet-shaped substrates coated with metal oxides, the metal oxide coating on the substrates can be titanium dioxide, iron oxides, chromium oxide, zinc oxide, silica, alumina, zirconium oxide, etc. The substrates can be mica, silica flakes, borosilicate flakes, alumina flakes, metal flakes, etc.

There are two advantages of using the silanes of the present invention. First, after hydrolysis, the silanol groups formed can react with the active sites of a pigment or hydroxyl groups of silica so that the silane is bonded strongly to the pigments. The second advantage is that in the case of quaternary amines, the pigments will have better adhesion to hair and skin because the quaternary amine is known for its good hair and skin adhesion properties. In other word, the adhesion is improved due to the attraction between the positive charges of a quaternary amine and the negative charges of hair or skin.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

### EXAMPLE 1

### General Procedure of Making the Silane Treated Pigments

Mix 100 g of pigment and 1200 mL of deionized water in a 2 liter beaker. Heat the slurry to about 75° C. Add sulfuric acid and/or to sodium hydroxide to adjust the pH to 6.0-6.5. Titrate the slurry with a water glass solution (1.5 g SiO2/50 mL) over a period of 60 minutes. Sulfuric acid is used to maintain the pH at 6.0-6.5. Stir for approximately 10 minutes. 6% silane based on the pigment weight is added dropwise to the slurry over 15 minutes with no pH adjustment. Stir the suspension for 30 minutes. Use sulfuric acid or sodium hydroxide solution to adjust the pH to 6.0-6.5. Stop the stirring and heating and allow the pigments to settle. Filter and wash the pigment with deionized water and then dry the pigment at 145-155° C. overnight. Sieve the dried pigment.

The following examples demonstrate that the pigments obtained by the current invention show improved adhesion to hair.

### EXAMPLE 2

### Evaluation

A gold pearlescent pigment was used in the test. This pigment is mica coated with titanium dioxide and iron oxide (Colorona Red Gold from EMD Chemicals Inc.). It was treated with N,N-dimethylaminopropyl trimethoxysilane according to the above mentioned procedure. The conditioner used is a commercial product (Infusium 23 Leave-In Treatment). Dark Auburn dyed hair (from Demeo Brothers) was used.

The test was carried out following the procedures detailed below.

1. Prepare the conditioner samples:

Two samples were prepared with untreated and silane-treated pigments. The pigment concentration is 6.55% in the conditioner.

2. The hair swatch samples were shampooed before the application of the conditioners. The conditioners (with treated and untreated pigments respectively) were massaged into the two wet hair strands. The conditioner with untreated pigment was applied to hair A. Hair B was applied with the conditioner containing treated pigment. After the hairs were dried, they were combed and weighed.

3. Visual Evaluation:

Hair B appeared to have slightly more gold color than hair A but the difference was not significant.

4. Shampoo the two treated hair stands by rinsing the hair strands in shampoo solutions for 2 minutes. Then rinse the hairs in deionized water and dry the hair strands overnight. Comb the hair strands.

5. Observation and evaluation:

Visual: Hair B appeared to have more gold color than hair A.

A measurement was performed with an X-rite® color analysis instrument. The results are listed below. (Only L, a, b values at 25° are listed, the reported values are an average of two measurements.)

L is lightness/darkness scale, a represents red/green shades and b represents yellow-blue shades. If an object has a higher L value, the object is lighter than the one with lower L value; a high positive a value indicates that the object is redder and a high positive b value indicates that the object is more yellow. The value difference for each parameter has to be more than 0.5 to 1 so that the color difference can be observed visually.

| | L | a | b |
|---|---|---|---|
| Hair A | 39.76 | 13.55 | 17.45 |
| Hair B | 41.68 | 12.52 | 19.81 |

6. Conclusion: When used in a leave-on conditioner, the silane-treated pigment has better affinity to the hair, i.e. it improves the hair substantivity of pigments.

A photograph showing the two hair strands is attached (photograph 1).

### EXAMPLE 3

### Evaluation

A silver white pearlescent pigment is used in this experiment. It is Timiron MP-149 Diamond Cluster (from EMD Chemicals Inc) and is titanium dioxide coated mica. It was treated with dimethylaminopropyl trimethoxysilane (CH₃)₂—N—(CH₂)₃—Si—(OCH₃)₃ according to the above mentioned procedure. A simplified hair conditioner was prepared according to the following table.

| INCI NAME | TRADE NAME/MANUFACTURER | % |
|---|---|---|
| Phase A | | |
| Water | Water, Demineralized | 86.96 |
| Hydroxyethycellulose | Cellosize PCG-10/Amerchol | 0.84 |
| Phase B | | |
| Propylene Glycol | Propylene Glycol/Lyondell | 1.50 |
| Methylparaben | Methylparaben/Spectrum Chemical | 0.20 |
| Phase C | | |
| Cetyl Alcohol | Lanette 16/Cognis | 2.00 |
| Cetearyl Alcohol (and) | Promulgen D/Amerchol | 4.50 |
| Ceteareth-20 | | |
| Phase D | | |
| Mica (and) Titanium | Timiron ® MP-149/Rona | 4.00 |
| Dioxide | | |
| Total | | 100.00 |

Procedure: Disperse Cellosize in water and heat to 750 with stirring. Blend ingredients in Phase B and add to Phase A. Heat Phase C to 80° C. with stirring. Add Phases AB slowly to Phase C with stirring. Cool down to 65° C. and homogenize. Adjust pH to 4.0-5.5 with TEA and/or citric acid, as needed. Blend in Phase D; mix until product is smooth and uniform.

The condition contains 4% treated or untreated titanium dioxide coated mica.

Two types of hair colors (auburn dyed hair and black dyed hair) were used in this test.

The hair swatches were immersed in 500 ml water solution (containing 3% cream PL-5-52A) for 3 min at room temperature.

After drying, the hairs were combed and evaluated visually. It was apparent that the hairs rinsed in the cream with treated pigment had more pigment particles attached than the hairs rinsed in the cream with untreated pigment. The photograph of the hair swatches were attached (photographs 2 and 3).

The entire disclosures of all applications, patents and publications, cited herein are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A surface treated pearlescent pigment obtained by a process comprising hydrolyzing an amine of the formulae
(R)₂N—(CH₂)ₙ—SIR′ R=alkyl or aryl
or
A⁻(R)₃—N—(CH₂)ₙ—SiR′ R′=alkoxy, C₁-C₃
n=3-5
A=an anion
to obtain silanol groups and contacting a pearlescent pigment with resultant hydrolyzate.

2. A hair-treatment composition comprising the resultant pigment of claim 1.

3. A surface treated pearlescent pigment according to claim 1, wherein the pigment is mica coated with titanium dioxide.

4. A surface treated pearlescent pigment according to claim 1, wherein the amine is dimethylaminopropyltrimethoxysilane.

5. A surface treated pearlescent pigment according to claim 3, wherein the amine is dimethylaminopropyltrimethoxysilane.

6. A method of providing substantivity to hair comprising applying a composition according to claim 2 to hair.
